(19) United States
(12) Patent Application Publication
(10) Pub. No.: US 2020/0215127 A1
(43) Pub. Date: Jul. 9, 2020

(54) TARGETED SYNBIOTIC THERAPY FOR DYSBIOSIS-RELATED INTESTINAL AND EXTRA-INTESTINAL DISORDERS

(71) Applicant: LifeBidge Health, Inc., Baltimore, MD (US)

(72) Inventor: Sudhir Kumar Dutta, Lutherville, MD (US)

(21) Appl. No.: 16/243,416

(22) Filed: Jan. 9, 2019

(51) Int. Cl. A61K 35/741; A61K 9/48; A61K 9/00; A61K 31/733; A61K 31/732; A61K 9/50

(52) U.S. Cl. CPC A61K 35/741; A61K 9/4891; A61K 9/50; A61K 31/733; A61K 31/732; A61K 9/0031

(57) ABSTRACT

Various types of synbiotic therapies are provided for the treatment of a variety of gastrointestinal and other disorders. The combination of prebiotics to probiotics is defined as a synbiotic therapy. The principal GI disorders associated with dysbiosis that can be treated from such a therapeutic intervention include but are not limited to: inflammatory bowel disease (IBD) (Crohn's disease and ulcerative colitis), irritable bowel syndrome (IBS), antibiotic-associated diarrheas such as recurrent Clostridium difficile infection, and possibly variants of Celiac disease. Other disorders that may also be ameliorated by the proposed synbiotic therapy include metabolic syndromes and central nervous system disorders. The disclosed methods and compositions were developed to improve upon currently available probiotics through consideration of the human intestinal microbiota, and its relationship to various intestinal metabolic and neuropsychiatric disorders. In one embodiment, the disclosed synbiotic compositions include prebiotics and a targeted delivery system, which altogether promote the survival, growth, and attachment of probiotic microbiota.

### CROSS REFERENCE TO RELATED APPLICATIONS

This application is a divisional of U.S. patent application Ser. No. 15/791,533 filed on Oct. 24, 2017 which claims benefit of and priority to U.S. Provisional Patent Applications 62/411,865 filed on Oct. 24, 2016, and 62/449,761 filed on Jan. 24, 2017, both of which are incorporated by reference in their entireties where permissible.

### FIELD OF THE INVENTION

The invention is generally directed to methods and compositions for modulating microbiota to treat dysbiosis or symptoms thereof.

### BACKGROUND OF THE INVENTION

Emerging scientific data suggest that human intestinal microbiota play a critical role in regulating host health. This system, known as the microbiome, encompasses trillions of non-pathogenic microorganisms of bacterial, Archaeal, viral, and fungal origins. The microbiome augments the host immune system to prevent the colonization of pathogenic microorganisms and regulates many essential metabolic functions by extracting otherwise inaccessible energy and nutrients from food that cannot be fully digested by host cells (Carding, et al., Microbial Ecology in Health and Disease, 26: 26191 (2015)). Microbial metabolites such as fermentation products or bacteriocins (narrow-spectrum antibiotic proteins produced by bacteria) have been implicated in this effect in addition to the activity of the microbes themselves. Evidence also suggests that the microbiota play yet another critical role in the pathogenesis of many diseases and disorders, leading to the conclusion that manipulating the microbiome may be crucial for the proper treatment of these afflictions.

The most compelling evidence for the link between microbiota and disease pathogenesis has been found in germ-free animal models for human autoimmune diseases, wherein exposure and colonization by microbes from the external environment is required for disease initiation and progression (Carding, et al., Microbial Ecology in Health and Disease, 26: 26191 (2015); Marchesi, et al., Gut, 1-10 (2015); Gkouskou, et al., Frontiers in Cellular and Infection Microbiology, 4: 28 (2014). Indeed, many human diseases and disorders have been associated with dysbiosis, or a disturbance within individual members or community structures in healthy commensal microbial populations (Carding, et al., Microbial Ecology in Health and Disease, 26: 26191 (2015)).

Inflammatory bowel diseases (IBD) such as Crohn's disease (CD) and ulcerative colitis (UC), as well as irritable bowel syndrome (IBS), Celiac disease, and antibiotic-associated diarrhea (AAD) have been linked to microbial dysbiosis in the GI tract (Carding, et al., Microbial Ecology in Health and Disease, 26: 26191 (2015); McHardy, et al., Microbiome, 1(1): 17 (2015)). Antibiotic-associated diarrheas are perhaps the most clear-cut examples of the relationship between dysbiosis and health. For example, Clostridium difficile infection (CDI) is one such disorder, wherein C. difficile bacteria overtake the colonic environment, leading to debilitating and sometimes deadly colitis. The symptoms of CDI have been associated with drastic changes in the diversity and community structure of the microbiome (Sangster, et al., Frontiers in Microbiology, 7: 789 (2016); Seekatz, et al., mBio, 5(3), e00893-14 (2014)). Metabolic diseases such as obesity and diabetes and even disorders of the central nervous system (CNS) have been linked to dysbiosis and will be referred to henceforth as “extra-intestinal disorders” (Carding, et al., Microbial Ecology in Health and Disease, 26: 26191 (2015)). The link between dysbiosis and many disorders has been established, but many questions remain in regards to the “healthy” microbiome, the mechanics behind dysbiosis, and their roles in disease pathogenesis.

The emerging age of culture-independent techniques (e.g., 16S rRNA analysis) has facilitated the observation of microbial communities and the determination of the specific microorganisms or microorganismal metabolites that may be harnessed to counteract dysbiosis. Results from studies relating to the healthy human microbiome and dynamics within the metabolic pathways in healthy and unhealthy subjects suggest the therapeutic potential of targeting the microbiome and even “mining” it for useful metabolites.

Previous attempts to manipulate the microbiome in order to counteract dysbiosis have been successful. Changes in diet are linked to changes in the microbial communities, which suggest that the microbiome can be manipulated directly for therapeutic purposes. One such manipulation is the method of fecal microbiota transplantation (FMT), wherein processed stool from a healthy donor is administered to an unhealthy subject. FMT represents a more direct manipulation of the microbiome, and has resulted in clear changes in the organization of the gut microbiota (Seekatz, et al., mBio, 5(3), e00893-14 (2014)). For example, stool transferred from lean to obese subjects has resulted in weight loss and observable changes to the microbiota (Hartstra, et al., Diabetes Care, 38(1), 159-165 (2015). FMT has also been studied extensively in CDI, and is currently the most effective method of treatment for recurrent CDI (Borody, et al., Current Gastroenterology Reports, 15: 8 (2013). The efficacy of this therapy is likely due to the presence of members of the healthy human microbiome in the administered fecal matter, which antagonize and outcompete pathogenic C. difficile and restore normal GI function by reclaiming metabolic niches. However, the use of this therapy is relatively new and concerns remain in regards to donor infection transmission, patient acceptance, and long-term effects within the recipient microbiome. Probiotics, or isolated colonies of microorganisms that confer benefits to the host have been demonstrated to have a similar effect on those affected by dysbiosis and eliminate the perceived risks associated with transferring fecal material between humans.

Probiotics are often encapsulated dormant microbial populations, which when administered are able to attach and populate the target anatomy in order to confer beneficial effects. However, even though there are hundreds of different probiotic products available on the market, few formulations extend beyond a small pool of microbiota, e.g. members of the Bifidobacteria and Lactobacillus genera. These products often do not involve a targeted delivery system, sufficient colony forming units (CFUs), or prebiotics, all of which are important components to ensure the survival and growth of the included microbes in the portion of the body they are intended for. In short, the field of probiotics requires improvements to effectively ameliorate gastrointestinal disorders caused by microbial dysbiosis.

The mechanism by which probiotic organisms may best improve host health is not well understood, but evidence suggests that the careful consideration of the metabolites known to play a critical role in human health is key in selecting probiotic organisms for the development of novel therapies (Larsen, et al., GigaScience, 4:42 (2015); McHardy, et al., Microbiome, 1(1): 17 (2015); The Human Microbiome Project Consortium, Nature, 486(7402): 207-214 (2013)).

As mentioned previously, microbes produce a multitude of metabolites, many of which have been identified as important to human host metabolic pathways (Donia, et al., Science, 349(6246): 1254766-1254766 (2015); The Human Microbiome Project Consortium, Nature, 486(7402): 207-214 (2013); Wong, et al., Journal of Clinical Gastroenterology, 40(3), 235-43 (2006). Microbial metabolites that influence GI health include short-chain fatty acids (SCFA), polyphenols, vitamins, and bacteriocins. SCFA are important examples of microbial metabolites that play a crucial role in the regulation of the healthy GI tract in humans.

Anaerobes of the human large intestine ferment polysaccharides, resulting in the production of three major SCFA: acetate, propionate, and butyrate (Hooper, et al., Annual Review of Nutrition, 22: 283-307 (2002)). SCFA contribute to host metabolism in addition to influencing colonic health through epithelial proliferation and differentiation (den Besten, et al., Journal of Lipid Research, 54(9): 2325-2340 (2013); Vinolo, et al., Nutrients, 3(12): 858-876 (2011); Wong, et al., Journal of Clinical Gastroenterology, 40(3), 235-43 (2006); Mortensen, et al., Scandinavian Journal Of Gastroenterology, 216: 132-148 (1996); Kripke, et al., Journal of Parenteral and Enteral Nutrition, 13: 109-116 (1989)). In fact, it is estimated that 60-75% of the energy derived from ingested carbohydrates can be attributed to SCFA production (Bergman, Physiol. Rev., 70:567-590 (1990)). Butyrate in particular is considered to have an important role in the regulation of digestive health (Pryde, et al., FEMS Microbiology Letters, 217(2): 133-139 (2002)).

Butyrate is an important example of a SCFA with a significant impact on host GI health. Colonic epithelial cells prefer butyrate as a food source; indeed, 70% to 90% of produced butyrate is metabolized by colonocytes (Wong, et al., Journal of Clinical Gastroenterology, 40(3), 235-43 (2006)), and overall the colonic epithelium fulfills 60-70% of its required energy from butyrate (Roediger, Gut, 21: 793-798 (1980)). In addition to its role in fueling colonic cells, butyrate is key in regulating cellular proliferation and differentiation (Wong, et al., Journal of Clinical Gastroenterology, 40(3), 235-43 (2006)). It has also been shown to produce anti-inflammatory effects by inhibiting the activation of transcription factor NF-KB, which leads to a reduction in proinflammatory cytokines.

The effect of butyrate in the colonic environment extends beyond regulating mucosal integrity. It has been shown that commensal gut microbiota regulate the immune system through their influence in the differentiation and development of a number of types of T cells. Further evidence indicates that it is the butyrate produced by these microbes (often bacteria) that plays this key role in the immune system, as butyrate and butyrate-producing microbes have been associated with the modulation of regulatory T cell (Tregs) differentiation and diversification (Furusawa, et al., Nature, 504(7480): 446-450 (2013). Though butyrate is considered a crucial metabolite, it is important to note that its production is strongly influenced by the presence of the other SCFAs (acetate and propionate) as well. Furthermore, there is still a great deal to discover in the field of the human microbial metabolome. All things considered, what is known in regards to microbial metabolites begs for the development of probiotics that will establish populations of microbes that produce these important agents.

Therefore, it is an object of the invention to provide compositions and methods for treating dysbiosis.

It is another object of the invention to provide probiotic and prebiotic compositions for the treatment of dysbiosis.

### SUMMARY OF THE INVENTION

Various types of synbiotic therapies are provided for the treatment of a variety of gastrointestinal and other disorders. The combination of prebiotics to probiotics is defined as a synbiotic therapy. The principal GI disorders associated with dysbiosis that can be treated from such a therapeutic intervention include but are not limited to: inflammatory bowel disease (IBD) (Crohn's disease and ulcerative colitis), irritable bowel syndrome (IBS), antibiotic-associated diarrheas such as recurrent Clostridium difficile infection, and possibly variants of Celiac disease. Other disorders that may also be ameliorated by the proposed synbiotic therapy include metabolic syndromes and central nervous system disorders. The disclosed methods and compositions were developed to improve upon currently available probiotics through consideration of the human intestinal microbiota, and its relationship to various intestinal metabolic and neuropsychiatric disorders. In one embodiment, the disclosed synbiotic compositions include prebiotics and a targeted delivery system, which altogether promote the survival, growth, and attachment of probiotic microbiota.

Another embodiment provides synbiotic compositions and methods that utilize healthy microbiota known to be present in the healthy human GI system, which also may produce important metabolites that may counteract dysbiosis. In addition, components likely to facilitate microbial attachment to intestinal mucosa can be included, known as prebiotics. Furthermore, the release of healthy microbes in the GI tract can be targeted towards specific portions of the anatomy (i.e., large intestine (colon), small intestine (jejunum and ileum), and/or stomach).

One embodiment provides compositions for treating dysbiosis containing viable non-pathogenic SCFA or other metabolite-producing microbes encapsulated within a targeted capsule in combination with prebiotics that facilitate microbial and host mucosal health. Representative commensal gut bacteria that produce SCFA include species in Clostridium, Eubacterium, Ruminococcus, Coprococcus, Dorea, Lachnospira, Roseburia and Butyrivibrio genera also known as Clostridium cluster IVXa. Another embodiment provides compositions containing species from Clostridium cluster IV which includes the Clostridium, Eubacterium, Ruminococcus and Anaerofilum genera. Representative SCFAs include acetate, propionate and butyrate.

In one embodiment, the viable non-pathogenic or attenuated microorganisms are derived from commercially-available cultures that have been carefully tested and proven to be non-pathogenic and/or attenuated and viable. The microorganisms are lyophilized and remain dormant until delivery to their target, and the conditions within the capsule will ensure their survival throughout the delivery and release processes.

Prebiotics that can be used in the disclosed compositions include, but are not limited to fructans including but not limited to inulin, trans-galactooligosaccharide, Larch arabinogalactin (LAG), polydextrose, psyllium, resistant starch, pectin, beta-glucans, Xylooligosaccharides (XOS), and combinations thereof. Additional substances that have prebiotics effects and can be included in the disclosed compositions include polydextrose, wheat dextrin, acacia gum, psyllium, banana, whole grain wheat, whole grain corn, and combinations thereof. An important mechanism of action for the prebiotics is fermentation of the prebiotics in the colon.

In some embodiments, the compositions are formulated to release their contents in the colon. In still other embodiments, the compositions are formulated with a targeting moiety to specifically direct the compositions to specific areas of the gastrointestinal tract, for example the colon.

Methods for treating dysbiosis include administering one more of the disclosed composition to subject in need thereof in an amount effective to modify the microbiome of subject and thereby treat the dysbiosis.

One embodiment provides a method for increasing production of SCFA in the gut of a subject in need thereof comprising administering to the subject an effective amount of the disclosed synbiotic compositions in increase production of acetate, propionate, butyrate or combinations thereof in the gut of the subject.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Compositions for Treating Dysbiosis

Dysbiosis is defined as an imbalance or disturbance within individual members or communities of microbial populations within the human gastrointestinal (GI) system. Elimination of dysbiosis has been associated with restoration of healthy GI microbiota, which in turn leads to improvements in GI disorders. Several lines of clinical and scientific evidence indicate that correction of microbial dysbiosis is feasible with the administration of probiotics and/or prebiotics. Probiotics are beneficial microbiota, which when administered promote health benefits. However, currently available probiotics in the market contain limited selections of microbes and do not include targeted delivery systems.

It has been discovered that dysbiosis can be treated using a combination of probiotics and prebiotics. One embodiment provides synbiotic compositions for treating dysbiosis containing a combination of probiotic microorganisms with prebiotic material in an amount to promote commensal microorganism growth in the gut of the subject or the production of metabolites from commensal microorganisms in the gut of the subject. Another embodiment provides synbiotic compositions containing an amount of prebiotic material to promote or induce adherence of the probiotic microorganisms to the mucosal lining of the gut. In still another embodiment the compositions are formulated for targeted delivery to the human gut.

A. Gut Commensal Microbiota

The human large intestine is one of the most diversely colonized and metabolically active organs in the human body. Up to 1000 different species of bacteria reside in the colon with microbial populations comprising approximately 10¹¹-10¹² cfu/g of contents. The colonic environment is favorable for bacterial growth due to its slow transit time, readily available nutrients, and favorable pH.

The disclosed synbiotic compositions contain commensal human gut microbiota. Human gut-associated microbiota are dominated by four main phyla: Firmicutes, Bacteroidetes, Actinobacteria and Proteobacteria and one or more members of these phyla maybe included in the disclosed compositions (Tlaskalová-Hogenová, H. et al., 2011). Other phyla that are less represented and may also be included in the disclosed synbiotic compositions include the Fusobacteria, Euryarchaeota, and Verrucomicrobia phyla.

The most abundant genera from the Bacteroidetes phylum are Bacteroides and Prevotella species, which represent 80% of all Bacteroidetes in fecal samples. Additional members of Bacteroidetes include, but are not limited to Bacteroides vulgatus, Barnesiella spp., and Odoribacter spp.

Representative members of Firmicutes include but are not limited to Anaerotruncus colihominis, Butyrivibrio crossotus, Clostridium spp., Caprococcos eutactus, Faecalibacterium prausnitzii, Lactobacillus spp., Pseudoflavonifractor spp., Roseburia spp., Ruminococcus spp., and Veillonella spp.

Representative members of the Actinobacteria phylum include but are not limited to Bifidobacterium spp., B. longum, and Collinsella aerofaciens.

A representative member of Euryarchaeota includes Methanobrevibacter smithii.

A representative member of Fusobacteria includes Fusobacterium spp.

A representative member of Verrucomicrobia includes Akkermansia muciniphila.

In one embodiment, the synbiotic composition includes one or more of the following:

Achromobacter spp., Actinomyces spp., Aeromonas spp., Acidaminococcus fermentans, Acinetobacter calcoaceticus, Akkermansia muciniphila, Alcaligenes faecalis, Anaerobiospirillum spp., Anaerotruncus colihominis, Bacillus spp., Bacteroides spp. including but not limited to Bacteroides Vulgatus, Bacteroides melaninogenicus and Bacteroides fragilis, Barnesiella spp., Bifidobacterium spp. including but not limited to Bifidobacterium longum, Butyrivibrio crossotus, Butyriviberio fibrosolvens, Campylobacter spp., Caprococcos eutactus, Clostridium spp. including but not limited to Clostridium difficile and Clostridium sordellii, Collinsella aerofaciens, Enterococcus spp., Eubacterium spp., Faecalibacterium prausnitzii, Flavobacterium spp., Fusobacterium spp., Lactobacillus spp., Methanobrevibacter smithii, Morganella morganii, Mycobacteria spp., Mycoplasma spp., Odoribacter spp., Peptococcus spp., Peptostreptococcus spp., Prevotella spp, Propionibacterium spp., Providencia spp., Pseudoflavonifractor spp., Pseudomonas aeruginosa, Roseburia spp., Ruminococcus spp. including but not limited to Ruminococcus bromii, Sarcina spp., Staphylococcus aureus, Streptococcus viridans, Yersinia enterocolitica, Veillonella spp., Vibrio spp., and combinations thereof.

In one embodiment, the synbiotic composition includes one or more species belonging to one or more genera or species selected from the group consisting of Bifidobacterium, Bacteroides, Tannerella, Parabacteroides, Bacillus, Lactobacillus, Anaerostipes, Anaerostipes, Blautia, Coprococcus, Dorea, Clostridium XI, Collinsella, and Paraprevotella. In still another embodiment, the synbiotic composition includes Clostridium sp., Lactobacillus sp., Lactobacillus murinus, Mucispirillum schaedleri, Eubacterium plexicaudatum, Firmicutes bacterium, Clostridium sp. and Parabacteroides sp. In another embodiment, the synbiotic composition includes Paraprevotella clara, Bifidobacterium longum, Collinsella aerofaciens, Coprococcus comes, Dorea longicatena, Bacteroides eggerthii str., and Bacteroides vulgates.

In still another embodiment, the synbiotic compositions include viable non-pathogenic SCFA or other metabolite-producing microbes encapsulated within a targeted capsule in combination with prebiotics that facilitate microbial and host mucosal health. Representative commensal gut bacteria that produce SCFA include species in Clostridium, Eubacterium, Ruminococcus, Coprococcus, Dorea, Lachnospira, Roseburia and Butyrivibrio genera also known as Clostridium cluster IVXa. Another embodiment provides compositions containing species from Clostridium cluster IV which includes the Clostridium, Eubacterium, Ruminococcus and Anaerofilum genera. Representative SCFAs include acetate, propionate and butyrate.

The number of microorganisms per dosage unit is typically 10⁵ to 10¹² colony forming units (CFU) depending upon formulation.

B. Prebiotics

As discussed above, the disclosed compositions also contain prebiotic material (also referred to as prebiotics). Prebiotics are non-digestible fiber that resists gastric acidity, hydrolysis by mammalian enzymes, and adsorption in the human upper gastrointestinal tract. Prebiotics are digestible by human intestinal microbiota and can stimulate the growth or activity or both of intestinal bacteria associated with health and well-being. In certain embodiments, prebiotics also facilitate microbial attachment to intestinal mucosa. Exemplary prebiotics that are included in the disclosed compositions include, but are not limited to fructans including but not limited to inulin, trans-galactooligosaccharide, Larch arabinogalactin (LAG), polydextrose, psyllium, resistant starch, pectin, beta-glucans, Xylooligosaccharides (XOS), and combinations thereof. Additional substances that have prebiotics effects that can be included in the disclosed compositions include polydextrose, wheat dextrin, acacia gum, psyllium, banana, whole grain wheat, and whole grain corn. An important mechanism of action for dietary fiber and prebiotics is fermentation in the colon and changes in gut microflora.

C. Intestinal Targeting

1. Enteric Formulations

The disclosed composition can be formulated to be released in specific locations of the GI tract. For example, the compositions can be targeted to the oral cavity, stomach, small intestine, ileum, or colon. To reach the colon and release the drug, a dosage form must be formulated taking into account various obstacles introduced by the gastrointestinal tract. Successful delivery of a drug to the colon requires protection of the drug from degradation or release in the stomach and then controlled release of drug in colon. The desired properties of colon targeted drug delivery systems can be achieved by using some polymers either alone or in a combination because it is now recognized that polymers can potentially influence the rate of release and absorption of drugs and play an important role in formulating colon targeted drug delivery systems.

Materials for intestinal targeting which can be used for surrounding or encapsulating the formulation are well known to a person skilled in the art. Preferably, the encapsulating material includes a compound which is insoluble in the gastrointestinal fluid at a pH of below 5 and which is soluble in the intestinal fluid at a pH at or above 5. Thus, this material dissolves in a pH dependent manner. The encapsulating material has a pH threshold which is the pH below which it is insoluble and at or above which it is soluble. The pH of the surrounding medium triggers the solution of the encapsulating material. Thus, none (or essentially none) of the encapsulating material dissolves below the pH threshold. Once the pH of the surrounding medium reaches (or exceeds) the pH threshold, the encapsulating material becomes soluble.

The term “insoluble” refers to when 1 g of the material requires more than 10,000 ml of solvent (surrounding medium) to dissolve at a given pH. By “soluble”, it is understood that 1 g of the material requires less than 10,000 ml, preferably less than 5,000 ml, more preferably less than 1,000 ml, even more preferably less than 100 ml or 10 ml of solvent to dissolve at a given pH. “Surrounding medium” means the medium in the gastrointestinal tract, such as the gastric fluid or intestinal fluid. Alternatively, the surrounding medium may be an in vitro equivalent of the medium in the gastrointestinal tract.

The normal pH of gastric fluid is usually in the range of 1 to 3. The material for intestinal, preferably colon targeting is insoluble below pH 5 and soluble at or above pH 5. The material therefore is usually insoluble in gastric fluid. Such material may be referred to as an “enteric” material. The pH of intestinal fluid gradually increases to about 7 to 8 along the small intestine. The material for intestinal targeting therefore becomes soluble in the terminal ileum/colon and allows release of the active agent from the composition. The material preferably has a pH threshold of 6.5, more preferably of 7.

Examples of suitable materials for intestinal targeting and in particular for the preparation of a coating surrounding the composition are gelatin, acrylate polymers, cellulose polymers and polyvinyl-based polymers, chitosan, its derivatives or other polymers. Examples of suitable cellulose polymers include cellulose acetate phthalate, cellulose acetate trimellitate and hydroxypropylmethyl cellulose acetate succinate. Examples of suitable polyvinyl-based polymers include polyvinylacetate phthalate.

In one embodiment the material for intestinal targeting can be a copolymer of a (meth)acrylic acid and a (meth)acrylic acid C₁₋₄ alkyl ester, for instance, a copolymer of methacrylic acid and methacrylic acid methyl ester. Suitable examples of such copolymers are usually anionic and not sustained release polymethacrylates. The ratio of carboxylic acid groups to methylester groups in these co-polymers determines the pH at which the copolymer is soluble. The acid:ester ratio may be from about 2:1 to about 1:3, e.g. about 1:1 or, about 1:2. The molecular weight of such anionic copolymers is usually from about 120,000 to 150,000, preferably about 135,000.

Known anionic poly(methycrylic acid/methyl methacrylate) co-polymers include Eudragit® L (pH threshold about 6.0), Eudragit® S (pH threshold about 7) and Eudragit® FS (pH threshold about 7). Eudragit® L 100-55 which is a copolymer of methacrylic acid and ethylacetate and which has a pH threshold of about 5.5 is also suitable. The Eudragit® copolymers can be obtained from Evonik.

In one embodiment, the polymer coating contains linear polysaccharides. Linear polysaccharides remain intact in stomach and small intestine but the bacteria of human colon degrades them and thus make them potentially useful in colon targeted drug delivery systems. Exemplary linear polysaccharides include, but are not limited to guar gum, pectin, chondroitin sulfate, dextran, chitosan, cyclodextrin, inulin, amylose, locust bean gum, and combinations thereof. Some of these polymers are also prebiotic material.

In addition or alternatively to the above described compounds having a pH threshold the material for intestinal, preferably colon targeting may comprise a compound which is susceptible to attack by colonic bacteria, such as polysaccharides. Suitable polysaccharides are for example starch, amylose, amylopectine, chitosan, chondroitine sulfate, cyclodextrine, dextrane, pullulan, carrageenan, scleroglucan, chitin, curdulan and levan.

2. Mucoadhesive Formulations

The disclosed compositions can also include a mucoadhesive agent or polymer. In the case of polymer attached to the mucin layer of a mucosal tissue, the term “mucoadhesion” is used. The mucosal layer lines a number of regions of the body including the gastrointestinal tract, the urogenital tract, the airways, the ear, nose and eye. Suitable polymers that can be used to form mucoadhesive compositions include soluble and insoluble, non-biodegradable and biodegradable polymers. Representative polymers that can be used to make mucoadhesive compositions include, but are not limited to hydrogels, thermoplastics, homopolymeres, copolymers or blends, and natural or synthetic polymers.

Exemplary hydrophilic polymers include but are not limited to methylcellulose, hydroxyethyl, cellulose, hydroxy propyl methyl cellulose, sodium carboxy methyl cellulose, carbomers, chitosan, plant gums, and combinations thereof.

Exemplary hydrogels include but are not limited to poly(acrylic acid co acrylamide) copolymers, carrageenan, sodium alginate, guar gum, modified guar gum, or combinations thereof.

Exemplary thermoplastic polymers include, but are not limited to non-erodible neutral polystyrene and semi crystalline bioerodible polymers, which generate the carboxylic acid groups as they degrade, e.g. polyanhydrides and polylactic acid. Various synthetic polymers used in mucoadhesive formulations include polyvinyl alcohol, polyamides, polycarbonates, polyalkylene glycols, polyvinyl ethers, esters and halides, polymethacrylic acid, polymethylmethacrylic acid, methylcellulose, hydroxypropyl cellulose, hydroxypropyl methylcellulose and sodium carboxymethylcellulose.

Various biocompatible polymers used in mucoadhesive formulations include cellulose-based polymers, ethylene glycol polymers and its copolymers, oxyethylene polymers, polyvinyl alcohol, polyvinyl acetate and esters of hyaluronic acid.

Various biodegradable polymers used in mucoadhesive formulations are poly(lactides), poly(glycolides), poly(lactide-co-glycolides), polycaprolactones, and polyalkyl cyanoacrylates. Polyorthoesters, polyphosphoesters, polyanhydrides, polyphosphazenes are the recent additions to the polymers.

3. Ligands for Targeted Delivery to the GI

The disclosed synbiotic compositions can be targeted to specific mucosal tissues. In some embodiments, the compositions have site specific agents anchored on the composition. The compositions can contain mucus or cell-specific ligands. Exemplary targeting agents include but are not limited to Galanthus nivalis agglutinin, wheat germ agglutinin, Lycopersicon esculentum or tomato lectin, Lectin ML01 from Visum album, Phaseolus vulgaris isoagglutinin, Aleuria aurentia agglutinin, Abrus precatroisu lectin, Agaricus bisporus lectin, Anguilla anguilla, Arachis hypogaea, Pandeiraea simplicifolia, Bauhinia pupurea, and combinations thereof.

In another embodiment the compositions can include bacterial adhesion factors such as fimbriae. An exemplary fimbriae is K99 fimbriae.

### III. Method of Use

The disclosed synbiotic compositions can be used to treat GI disorders as well as metabolic syndromes and central nervous system disorders.

One embodiment provides a method for treating a GI disorder by administering to a subject in need thereof an effective amount of one or more of the disclosed synbiotic compositions. Representative GI disorders that can be treated include, but are not limited to inflammatory bowel disease, Crohn's disease, ulcerative colitis, irritable bowel syndrome, Celiac disease, small intestinal bacterial overgrowth (SIBO), antibiotic-associated diarrhea (AAD), and Clostridium difficile related diarrhea.

Another embodiment provides a method for treating a metabolic disorder by administering one or more of the disclosed synbiotic compositions to a subject in need thereof. Such disorders include, but are not limited to diabetes, bacterial dysbiosis, hyperlipidemia, metabolic syndrome, obesity and carbohydrate intolerance.

Still another embodiment provides a method for treating a central nervous system disorder by administering an effective amount of one or more of the disclosed synbiotic compositions. Such disorders may include, but are not limited to autism spectrum disorder, Parkinson's disease and multiple sclerosis.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The disclosed compositions can be administered orally, rectally, or into surgical pouches. The typical dose will range from 1-4 dosage units for up to 4 times per day for up to 4 weeks.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A method for treating dysbiosis in a subject in need thereof comprising administering to the subject a synbiotic composition comprising viable, non-pathogenic human gut microbes in combination with prebiotic material.

2. The method of claim 1, wherein the subject has inflammatory bowel disease (IBD), Crohn's disease (CD), ulcerative colitis (UC), irritable bowel syndrome (IBS), Celiac disease, or antibiotic-associated diarrhea (AAD).

3. A method for treating a disorder of the gastrointestinal tract comprising administering a synbiotic composition comprising viable, non-pathogenic human gut microbes in combination with prebiotic material.

4. The method of claim 3, wherein the disorder is selected from the group consisting of inflammatory bowel disease (IBD), Crohn's disease (CD), ulcerative colitis (UC), irritable bowel syndrome (IBS), Celiac disease, antibiotic-associated diarrhea (AAD), or Clostridium difficile related diarrhea.

5. (canceled)

6. The method of claim 1, wherein the composition is administered orally.

7. The method of claim 1, wherein the composition is administered rectally.

8. The method of claim 1, wherein the composition is administered directly into a surgical pouch.

9. The method of claim 1, wherein the microbes produce metabolites that improve or promote colon health.

10. The method of claim 1, wherein the prebiotic material comprises inulin, pectin, or a combination thereof.

11. The method of claim 1, wherein the prebiotic material facilitates microbial attachment to gut mucosa and facilitates overall growth and survival of gut microbiota.

12. The method of claim 1, wherein the synbiotic composition is formulated as an enteric coated capsule, or an enteric coated microcapsule for oral administration.

13. The method of claim 1, wherein the synbiotic composition releases its contents exclusively upon reaching a specific target portion of the gastrointestinal tract.

14. The method of claim 1, wherein the microbes are inactive until their release within the gastrointestinal tract.

15. The method of claim 3, wherein the microbes produce metabolites that improve or promote colon health.

16. The method of claim 3, wherein the prebiotic material comprises inulin, pectin, or a combination thereof.

17. The method of claim 3, wherein the prebiotic material facilitates microbial attachment to gut mucosa and facilitates overall growth and survival of gut microbiota.

18. The method of claim 3, wherein the synbiotic composition is formulated as an enteric coated capsule, or an enteric coated microcapsule for oral administration.

19. The method of claim 3, wherein the synbiotic composition releases its contents exclusively upon reaching a specific target portion of the gastrointestinal tract.

20. The method of claim 3, wherein the microbes are inactive until their release within the gastrointestinal tract.
